(19) United States
(12) Patent Application Publication
(10) Pub. No.: US 2025/0178028 A1
(43) Pub. Date: Jun. 5, 2025

(54) ULTRASONIC TRANSMITTING CIRCUIT, ULTRASONIC IMAGING APPARATUS, AND METHOD FOR GENERATING EXCITATION SIGNAL

(71) Applicant: SHENZHEN MINDRAY BIO-MEDICAL ELECTRONICS CO., LTD., Shenzhen (CN)

(72) Inventors: Qun Li HU, Shenzhen (CN); Rui Hu, Shenzhen (CN)

(21) Appl. No.: 19/049,973

(22) Filed: Feb. 10, 2025

Related U.S. Application Data
(63) Continuation of application No. PCT/CN2023/111556, filed on Aug. 7, 2023.

(30) Foreign Application Priority Data
Aug. 12, 2022 (CN) 202210969917.7

(51) Int. Cl. B06B 1/02; A61B 8/00; H01F 27/28; H03K 17/687

(52) U.S. Cl. CPC B06B 1/0215; A61B 8/54; H01F 27/28; H03K 17/6872

(57) ABSTRACT

The embodiments of the present disclosure provide ultrasonic transmitting circuits and ultrasonic imaging apparatuses. The ultrasonic transmitting circuit may include a transformer, a first input circuit and a second input circuit. The first input circuit is connected to the first end of the primary winding of the transformer. The second input circuit is connected to the second end of the primary winding of the transformer. The first end of the secondary winding of the transformer is connected to the output end of the ultrasonic transmitting circuit. The transformer outputs an excitation signal at the first end of its secondary winding according to the signals inputted by the first input circuit and the second input circuit at the primary winding.

### CROSS-REFERENCE TO RELATED APPLICATIONS

This application is a continuation application of International Patent Application No. PCT/CN2023/111556, filed on Aug. 7, 2023, which is based on and claims priority to and benefits of Chinese Patent Application No. 202210969917.7 filed on Aug. 12, 2022. The entire content of all of the above-referenced applications is incorporated herein by reference.

### TECHNICAL FIELD

The present disclosure relates to medical ultrasonic imaging, in particular to an ultrasonic transmitting circuit, an ultrasonic imaging apparatus and a method for generating excitation signals for exciting an ultrasonic probe to generate ultrasonic waves.

### BACKGROUND

Ultrasonic transmitting is an important part of ultrasonic imaging, in which multi-channel specific delay sequence/specific excitation waveform are generated to excite the transducers in the ultrasonic probe to transmit ultrasonic waves.

In an ultrasonic imaging apparatus, the controller can generate the transmitting control signal according to the object to be scanned by the ultrasonic probe, the scanning frequency, the depth or other information. The transmitting control signal can control the transmitting circuit to generate a high voltage transmitting waveform (i.e., an excitation signal). The performance of the generated high voltage transmitting waveform is one of the important factors determining the quality of the ultrasonic images.

In some important ultrasonic imaging modes, such as the harmonic imaging mode or the contrast enhanced imaging mode, the ultrasonic probe needs to be excited to transmit a symmetrical waveform, such as a waveform with a phase difference of 180 degrees. In this case, the symmetry is an important indicator of the high voltage transmitting waveform, and is of great significance in the imaging modes such as the harmonic imaging mode and the contrast enhanced imaging mode. The symmetry of the transmitting waveform directly affects the imaging performance of these imaging modes.

In an existing transmitting circuit, whether it is a pulse transmitting circuit or an arbitrary waveform transmitting circuit, the positive level is usually realized by PMOS transistor, and the negative level is usually realized by NMOS transistor. In the current semiconductor process, the PMOS transistor and the NMOS transistor themselves are components of two processes, and the parameter performance thereof cannot be exactly the same. Therefore, in practice, the positive and negative levels generated by the PMOS transistor and NMOS transistor cannot be well symmetrical. Actually, the symmetry of the transmitting realized by the existing circuits is difficult to exceed 45 db, and is generally around 40 db.

### SUMMARY

Embodiments of the present disclosure provide ultrasonic transmitting circuits, ultrasonic imaging apparatuses and methods for generating excitation signals for exciting an ultrasonic probe to generate ultrasonic waves. The excitation signals generated by the circuits and methods have better symmetry.

In one embodiment, an ultrasonic imaging apparatus is provided, which may include:
an ultrasonic probe including a transducer;
a transformer including at least a primary winding and a secondary winding, where, the primary winding includes a first end and a second end, the secondary winding include a first end, and the first end of the secondary winding is connected to the transducer of the ultrasonic probe;
a first input circuit connected to the first end of the primary winding; and
a second input circuit connected to the second end of the primary winding;
where, the transformer outputs an excitation signal at the first end of the secondary winding according to a signal inputted by the first input circuit at the first end of the primary winding and a signal inputted by the second input circuit at the second end of the primary winding, where the excitation signal excites the transducer connected to the first end of the secondary winding to transmit ultrasonic waves to a target object.

In one embodiment, the first input circuit inputs a first input signal at the first end of the primary winding at a first time, the second input circuit inputs a second input signal at the second end of the primary winding at the first time, and the transformer outputs a first excitation signal at the first end of the secondary winding according to the first input signal inputted at the first end of the primary winding and the second input signal inputted at the second end of the primary winding; and
the first input circuit inputs a third input signal at the first end of the primary winding at a second time, the second input circuit inputs a fourth input signal at the second end of the primary winding at the second time, and the transformer outputs a second excitation signal at the first end of the secondary winding according to the third input signal inputted at the first end of the primary winding and the fourth input signal inputted at the second end of the primary winding;
where, the first input signal is the same as the fourth input signal, and the second input signal is the same as the third input signal.

In one embodiment, the first input signal is different from the second input signal.

In one embodiment, the devices for implementing the second input circuit are the same as the devices for implementing the first input circuit.

In one embodiment, the ultrasonic imaging apparatus may further include a first resistor. One end of the first resistor is connected to the first end of the primary winding, and the other end of the first resistor is grounded.

In one embodiment, the ultrasonic imaging apparatus may further include a second resistor. One end of the second resistor is connected to the second end of the primary winding, and the other end of the second resistor is grounded.

In one embodiment, the ultrasonic imaging apparatus may further include a first capacitor. One end of the first capacitor is connected to the first end of the primary winding, and the other end of the first capacitor is grounded.

In one embodiment, the first input circuit may include:
a first analog-to-digital converter that receives a transmitting driving signal through an input end thereof and converts the transmitting driving signal into a digital driving signal; and
a first amplifier, where, an input end of the first amplifier is connected to an output end of the first analog-to-digital converter, an output end of the first amplifier is connected to the first end of the primary winding, and the first amplifier amplifies the digital driving signal outputted by the first analog-to-digital converter and inputs the amplified digital driving signal to the first end of the primary winding.

In one embodiment, the second input circuit may include:
a second analog-to-digital converter that receives a transmitting driving signal through an input end thereof and converts the transmitting driving signal into a digital driving signal; and
a second amplifier, where, an input end of the second amplifier is connected to an output end of the second analog-to-digital converter, an output end of the second amplifier is connected to the second end of the primary winding, and the second amplifier amplifies the digital driving signal outputted by the second analog-to-digital converter and inputs the amplified digital driving signal to the second end of the primary winding.

In one embodiment, the second analog-to-digital converter is the same device as the first analog-to-digital converter, and the second amplifier is the same device as the first amplifier.

In one embodiment, the first input circuit may include:
a first switch comprising a first end, a second end and a control end, where, the first end of the first switch is connected to a first high voltage end that receives a first high voltage signal, the second end of the first switch is connected to the first end of the second switch and is connected to the first end of the primary winding of the transformer, and the control end of the first switch receives a first control signal; and
a second switch comprising a first end, a second end and a control end, where, the second end of the second switch is connected to a first low voltage end that receives a first low voltage signal, and the control end of the second switch receives a second control signal.

In one embodiment, the second input circuit may include:
a third switch comprising a first end, a second end and a control end, where, the first end of the third switch is connected to a second high voltage end that receives a second high voltage signal, the second high voltage signal is the same as the first high voltage signal, the second end of the third switch is connected to the first end of the fourth switch and is connected to the second end of the primary winding of the transformer, and the control end of the third switch receives a third control signal; and
a fourth switch comprising a first end, a second end and a control end, where, the second end of the fourth switch is connected to a second low voltage end that receives a second low voltage signal, the second low voltage signal is the same as the first low voltage signal, and the control end of the fourth switch receives a fourth control signal.

In one embodiment, the third switch is the same device as the first switch, and the fourth switch is the same device as the second switch.

In one embodiment, the first switch is an NMOS transistor, the first end of the first switch is a drain of the NMOS transistor, the second end of the first switch is a source of the NMOS transistor, and the control end of the first switch is a gate of the NMOS transistor; and
the second switch is a PMOS transistor, the first end of the second switch is a drain of the PMOS transistor, the second end of the second switch is a source of the PMOS transistor, and the control end of the second switch is a gate of the PMOS transistor.

In one embodiment, the third switch is an NMOS transistor, the first end of the third switch is a drain of the NMOS transistor, the second end of the third switch is a source of the NMOS transistor, and the control end of the third switch is a gate of the NMOS transistor; and
the fourth switch is a PMOS transistor, the first end of the fourth switch is a drain of the PMOS transistor, the second end of the fourth switch is a source of the PMOS transistor, and the control end of the fourth switch is a gate of the PMOS transistor.

In one embodiment, the first switch is a PMOS transistor, the first end of the first switch is a drain of the PMOS transistor, the second end of the first switch is a source of the PMOS transistor, and the control end of the first switch is a gate of the PMOS transistor; and
the second switch is an NMOS transistor, the first end of the second switch is a drain of the NMOS transistor, the second end of the second switch is a source of the NMOS transistor, and the control end of the second switch is a gate of the NMOS transistor.

In one embodiment, the third switch is a PMOS transistor, the first end of the third switch is a drain of the PMOS transistor, the second end of the third switch is a source of the PMOS transistor, and the control end of the third switch is a gate of the PMOS transistor; and
the fourth switch is an NMOS transistor, the first end of the fourth switch is a drain of the NMOS transistor, the second end of the fourth switch is a source of the NMOS transistor, and the control end of the fourth is a gate of the NMOS transistor.

In one embodiment, the first switch is a PNP triode transistor, the first end of the first switch is a collector of the PNP triode transistor, the second end of the first switch is an emitter of the PNP triode transistor, and the control end of the first switch is a base of the PNP triode transistor; and
the second switch is an NPN triode transistor, the first end of the second switch is a collector of the NPN triode transistor, the second end of the second switch is an emitter of the NPN triode transistor, and the control end of the second switch is a base of the NPN triode transistor.

In one embodiment, the third switch is a PNP triode transistor, the first end of the third switch is a collector of the PNP triode transistor, the second end of the third switch is an emitter of the PNP triode transistor, and the control end of the third switch is a base of the PNP triode transistor; and
the fourth switch is an NPN triode transistor, the first end of the fourth switch is a collector of the NPN triode transistor, the second end of the fourth switch is an emitter of the NPN triode transistor, and the control end of the fourth switch is a base of the NPN triode transistor.

In one embodiment, the first switch is an NPN triode transistor, the first end of the first switch is a collector of the NPN triode transistor, the second end of the first switch is an emitter of the NPN triode transistor, and the control end of the first switch is a base of the NPN triode transistor; and
the second switch is a PNP triode transistor, the first end of the second switch is a collector of the PNP triode transistor, the second end of the second switch is an emitter of the PNP triode transistor, and the control end of the second switch is a base of the PNP triode transistor.

In one embodiment, the third switch is an NPN triode transistor, the first end of the third switch is a collector of the NPN triode transistor, the second end of the third switch is an emitter of the NPN triode transistor, and the control end of the third switch is a base of the NPN triode transistor; and
the fourth switch is a PNP triode transistor, the first end of the fourth switch is a collector of the PNP triode transistor, the second end of the fourth switch is an emitter of the PNP triode transistor, and the control end of the fourth switch is a base of the PNP triode transistor.

In one embodiment, the ultrasonic imaging apparatus may further include:
a receiving circuit configured to control the ultrasonic probe to receive ultrasonic echoes returned from the target object to obtain ultrasonic echo signals;
a processor configured to process the ultrasonic echo signals to obtain an ultrasonic image of the target object; and
a display configured to display the ultrasonic image.

In one embodiment, an ultrasonic transmitting circuit is provided, which may include:
a transformer comprising at least a primary winding and a secondary winding, where, the primary winding comprises a first end and a second end, the secondary winding comprises a first end, and the first end of the secondary winding is connected to a transducer of an ultrasonic probe;
a first input circuit that is connected to the first end of the primary winding; and
a second input circuit that is connected to the second end of the primary winding;
where, the transformer outputs an excitation signal at the first end of the secondary winding according to a signal inputted by the first input circuit at the first end of the primary winding and a signal inputted by the second input circuit at the second end of the primary winding, where the excitation signal excites the transducer connected to the first end of the secondary winding to transmit ultrasonic waves to a target object.

In one embodiment, a method for generating an excitation signal with an ultrasonic transmitting circuit is provided, where:
the ultrasonic transmitting circuit includes:
a transformer comprising at least a primary winding and a secondary winding, where, the primary winding comprises a first end and a second end, the secondary winding comprises a first end, and the first end of the secondary winding is connected to the transducer of the ultrasonic probe;
a first input circuit connected to the first end of the primary winding; and
a second input circuit connected to the second end of the primary winding;
where, the transformer outputs an excitation signal at the first end of the secondary winding according to a signal inputted by the first input circuit at the first end of the primary winding and a signal inputted by the second input circuit at the second end of the primary winding;
the method includes:
controlling the first input circuit to input a first input signal at the first end of the primary winding at a first time, and controlling the second input circuit to input a second input signal at the second end of the primary winding at the first time, such that the transformer outputs a first excitation signal at the first end of the secondary winding according to the first input signal inputted at the first end of the primary winding and the second input signal inputted at the second end of the primary winding; and
controlling the first input circuit to input a third input signal at the first end of the primary winding at a second time, and controlling the second input circuit to input a fourth input signal at the second end of the primary winding at the second time, such that the transformer outputs a second excitation signal at the first end of the secondary winding according to the third input signal inputted at the first end of the primary winding and the fourth input signal inputted at the second end of the primary winding;
where, the first input signal is the same as the fourth input signal, and the second input signal is the same as the third input signal.

In one embodiment, an ultrasonic transmitting circuit is provided, which may include:
a transformer comprising at least a primary winding and a secondary winding, where, the primary winding comprises a first end and a second end, the secondary winding comprises a first end, and the first end of the secondary winding is connected to a transducer of an ultrasonic probe;
a first input circuit connected to the first end of the primary winding, where the first input circuit comprises:
a first switch comprising a first end, a second end and a control end, where, the first end of the first switch is connected to a first high voltage end that receives a first high voltage signal, the second end of the first switch is connected to the first end of the second switch and is connected to the first end of the primary winding of the transformer, and the control end of the first switch receives a first control signal; and
a second switch comprises a first end, a second end and a control end, where, the second end of the second switch is connected to a first low voltage end that receives a first low voltage signal, and the control end of the second switch receives a second control signal; and
a second input circuit connected to the second end of the primary winding, where the second input circuit comprises:
a third switch comprising a first end, a second end and a control end, where, the first end of the third switch is connected to a second high voltage end that receives a second high voltage signal, the second high voltage signal is the same as the first high voltage signal, the second end of the third switch is connected to the first end of the fourth switch and is connected to the second end of the primary winding of the transformer, and the control end of the third switch receives a third control signal; and
a fourth switch comprising a first end, a second end and a control end, where, the second end of the fourth switch is connected to a second low voltage end that receives a second low voltage signal, the second low voltage signal is the same as the first low voltage signal, and the control end of the fourth switch receives a fourth control signal;
where, the transformer outputs an excitation signal at the first end of the secondary winding according to a signal inputted by the first input circuit at the first end of the primary winding and a signal inputted by the second input circuit at the second end of the primary winding, wherein the excitation signal excites the transducer connected to the first end of the secondary winding to transmit ultrasonic waves to a target object.

In one embodiment, a method for generating an excitation signal for exciting an ultrasonic probe to generate ultrasonic waves with an ultrasonic transmitting circuit described above, where the method include:
controlling the first switch to turn on, the second switch to turn off, the third switch to turn off and the fourth switch to turn on, such that a passage from the first high voltage end to the second low voltage end through the first switch, the primary winding of the transformer and the fourth switch is turned on, thereby outputting a first excitation signal at the first end of the secondary winding of the transformer; and
controlling the first switch turn off, the second switch to turn on, the third switch to turn on and the fourth switch to turn off, such that a passage from the first low voltage end to the second high voltage end through the second switch, the primary winding of the transformer and the third switch is turned on, thereby outputting a second excitation signal at the first end of the secondary winding of the transformer.

In the embodiments of the present disclosure, the first excitation signal is generated by the transformer according to the signal generated by the first input circuit and the signal generated by the second input circuit, and the second excitation signal is generated by the transformer according to the signal generated by the first input circuit and the signal generated by the second input circuit. That is, the first excitation signal is generated by the transformer, the first input circuit and the second input circuit, and the second excitation signal is also generated by the transformer, the first input circuit and the second input circuit. Therefore, the first excitation signal and the second excitation signal are generated by the same devices. Accordingly, the first excitation signal and the second excitation signal generated in the embodiments of the present disclosure have better symmetry than those generated by the conventional ultrasonic transmitting circuit.

### BRIEF DESCRIPTION OF THE DRAWINGS

FIG. 1 is a schematic block diagram of an ultrasonic imaging apparatus in one embodiment of the present disclosure;

FIG. 2 is a schematic block diagram of an ultrasonic transmitting circuit in one embodiment of the present disclosure;

FIG. 3 is a schematic diagram of an ultrasonic transmitting circuit in one embodiment of the present disclosure;

FIG. 4 is a schematic diagram of an ultrasonic transmitting circuit in one embodiment of the present disclosure;

FIG. 5 is a schematic diagram of an ultrasonic transmitting circuit in one embodiment of the present disclosure;

FIG. 6 is a schematic diagram of an ultrasonic transmitting circuit in one embodiment of the present disclosure;

FIG. 7 is a schematic diagram of an ultrasonic transmitting circuit in one embodiment of the present disclosure; and

FIG. 8 is a schematic diagram of an existing ultrasonic transmitting circuit.

### DETAILED DESCRIPTION

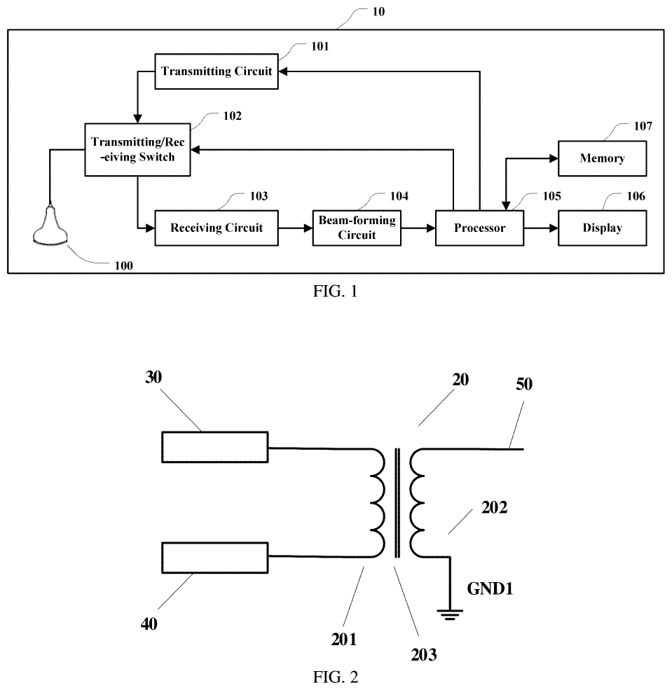

FIG. 1 is a schematic block diagram of an ultrasonic imaging apparatus in one embodiment of the present disclosure. The ultrasonic imaging apparatus 10 may include an ultrasonic probe 100, a transmitting circuit 101, a transmitting/receiving switch 102, a receiving circuit 103, a beam-forming circuit 104, a processor 105 and a display 106. The ultrasonic transmitting circuit 101 may generate excitation signals to excite the transducer of the ultrasonic probe 100 to transmit ultrasonic waves to the target tissue. The receiving circuit 103 may receive ultrasonic echoes returned from the target tissue through the ultrasonic probe 100, thereby obtaining ultrasonic echo signals/data. The ultrasonic echo signals/data may be sent to the processor 105 after the beam-forming processing is performed thereon by the beam-forming circuit 104. The processor 105 may process the ultrasonic echo signals/data to obtain the ultrasonic image of the target tissue. The ultrasound image obtained by the processor 105 may be stored in a memory 107. The ultrasound image may be displayed on the display 106.

In one embodiment of the present disclosure, the display 106 of the ultrasonic imaging apparatus 10 may be a touch screen, a liquid crystal display, etc., or an independent display device such as a liquid crystal display or a television independent of the ultrasonic imaging apparatus 10. Alternatively, it may also be a display screen on an electronic device such as a mobile phone or a tablet computer, and the like.

In practice, the processor 105 may be at least one of an Application Specific Integrated Circuit (ASIC), a Digital Signal Processor (DSP), a Digital Signal Processing Device (DSPD), a Programmable Logic Device (PLD), a Field Programmable Gate Array (FPGA), a Central Processing Unit (CPU), a controller, a microcontroller, a microprocessor, such that the processor 105 may perform the corresponding steps of the ultrasonic imaging method in various embodiments of the present disclosure.

The memory 107 may be a volatile memory, such as Random Access Memory (RAM); or a non-volatile memory, such as a Read Only Memory (ROM), a flash memory, a Hard Disk Drive (HDD) or a Solid-State Drive (SSD); or a combination of the memories above, and may provide instructions and data to the processor.

The ultrasonic transmitting circuit 101 may generate an excitation signal, which may be sent to the ultrasonic probe 100 to excite the transducer in the ultrasonic probe 100 to generate ultrasonic waves. FIG. 2 is a schematic diagram of the ultrasonic transmitting circuit in one embodiment of the present disclosure. As shown in FIG. 2, in this embodiment, the ultrasonic transmitting circuit may include a transformer 20, a first input circuit 30 and a second input circuit 40.

The transformer 20 may include at least a primary winding 201 and a secondary winding 202. The primary winding 201 may include a first end (e.g., the upper end in FIG. 2) and a second end (e.g., the lower end in FIG. 2). The secondary winding 202 may include a first end (e.g., the upper end in FIG. 2) and a second end (e.g., the lower end in FIG. 2). The transformer 20 may also include an iron core 203. It should be noted that the term “iron core” herein refers to the conventional name in the industry for this particular component in the transformer, but not mean that the component must be made of “iron”.

The transformation ratio of the transformer 20 may be determined according to actual needs, which may be any suitable value. For example, in one embodiment, the transformation ratio of the transformer 20 may be 1:1 or other suitable value.

The first input circuit 30 is connected to the first end of the primary winding 201 of the transformer 20, and can input a signal to the first end of the primary winding 201. The second input circuit 40 is connected to the second end of the primary winding 201 of the transformer 20, and can input a signal to the second end of the primary winding 201. The transformer 20 may output an excitation signal at the first end of the secondary winding 202 according to the signal input by the first input circuit 30 at the first end of the primary winding 201 and the signal input by the second input circuit 40 at the second end of the primary winding 201.

The first end of the secondary winding 202 of the transformer 20 is connected to the transducer of the ultrasonic probe. The excitation signal output from the first end of the secondary winding 202 may be transmitted to the transducer connected to the first end of the secondary winding 202, thereby exciting the transducer to transmit ultrasonic waves to the target object. Herein, the connection of the first end of the secondary winding 202 to the transducer of the ultrasonic probe may be a direct connection or an indirect connection through one or more other elements (e.g., as shown in FIG. 1, through the transmitting/receiving switch 102, etc.).

In this embodiment, the first input circuit 30 may input a first input signal S1 at the first end of the primary winding 201 at a first time, and the second input circuit 40 may input a second input signal S2 at the second end of the primary winding 201 at this first time. In this case, the transformer 20 may output a first excitation signal E1 at the first end of the secondary winding 202 according to the first input signal S1 and the second input signal S2. For example, assuming that the electrical parameter (for example, voltage, etc.) of the first input signal S1 is X and the electrical parameter of the second input signal S2 is Y, and assuming that the transformation ratio of the transformer 20 is 1:1, the electrical parameter of the excitation signal E1 output at the first end of the secondary winding 202 will be X-Y according to the nature of the transformer.

Then, at a second time different from the first time (e.g., the second time may immediately follow the first time, or the second time may be another time as required), the first input circuit 30 may input a third input signal S3 at the first end of the primary winding 201, and the second input circuit 40 may input a fourth input signal S4 at the second end of the primary winding 201. The transformer 20 may output a second excitation signal E2 at the first end of the secondary winding 202 according to the third input signal S3 and the fourth input signal S4. Herein, the first input signal S1 may be the same as the fourth input signal S4, and the second input signal S2 may be the same as the third input signal S3. In this case, for example, the electrical parameter (e.g., voltage, etc.) of the third input signal S3 is Y (the same as the second input signal S2), the electrical parameter of the fourth input signal S4 is X (the same as the first input signal S1), and the transformer 20 is assumed to be 1:1 as mentioned above. Therefore, according to the nature of the transformer, the electrical parameter of the excitation signal E2 output at the first end of the secondary winding 202 will be Y-X, which is −(X-Y). That is, E2=−E1. In this way, the transmission of symmetrical waveforms is realized, such as the transmission of positive level (for example, X-Y) and negative level (for example, −(X-Y)).

In this disclosure, two or more signals being the “same” may mean that the two or more signals are themselves the same one signal (but connected to different components or different locations), or that the two or more signals have at least partially the same signal parameter (e.g., voltage, current, phase or frequency, etc.). The “same” herein is not strictly limited to the absolute same in the mathematical sense, but may have certain errors in practice.

Usually, to achieve the symmetrical transmitting with better symmetry, it is desired that the first excitation signal and the second excitation signal have better symmetry. In a conventional ultrasonic transmitting circuit (as shown in FIG. 8), when a symmetrical transmitting is performed, the first excitation signal (for example, positive level) is usually generated by the element Q9 (PMOS transistor), and the corresponding second excitation signal (for example, negative level) of the symmetrical transmitting is generated by the element Q10 (NMOS transistor), so as to excite the transducer of the ultrasonic probe to transmit symmetrically. In this conventional ultrasonic transmitting circuit, the first excitation signal and the second excitation signal are respectively generated by different devices (Q9 and Q10). Since the two devices are devices made by different processes, their parameter performance cannot be completely consistent. Therefore, the symmetry of the first excitation signal and the second excitation signal generated by them respectively will be relatively poor.

In the embodiments of the present disclosure, as described above, the first excitation signal E1 is generated by the transformer 20 according to the signal X generated by the first input circuit 30 and the signal Y generated by the second input circuit 40, and the second excitation signal E2 is generated by the transformer 20 according to the signal Y generated by the first input circuit 30 and the signal X generated by the second input circuit 40. That is, the first excitation signal E1 is generated by the transformer 20, the first input circuit 30 and the second input circuit 40, and the second excitation signal E2 is also generated by the transformer 20, the first input circuit 30 and the second input circuit 40. Accordingly, the first excitation signal E1 and the second excitation signal E2 are generated by the same devices. Therefore, the first excitation signal E1 and the second excitation signal E2 generated in the embodiments of the present disclosure have better symmetry than the conventional ultrasonic transmitting circuit.

In the embodiments of the present disclosure, the first input signal S1 and the second input signal S2 are different signals, and the third input signal S3 and the fourth input signal S4 are different signals. Herein, two or more signals being “different” may mean that at least part of the parameters (e.g., voltage, current, phase, frequency, etc.) of said two or more signals are different. For example, the first input signal S1 and the second input signal S2 may be different in the phase by a predetermined angle, such as 180 degrees or other degrees. Alternatively, the first input signal S1 and the second input signal S2 may have different voltages, and so on.

In some embodiments of the present disclosure, the devices for implementing the second input circuit 40 may be the same as the devices for implementing the first input circuit 30. In this way, the consistency between the devices for generating the first excitation signal E1 and the second excitation signal E2 can be further improved. Therefore, the symmetry of the generated first excitation signal E1 and the second excitation signal E2 can be further improved. Herein, the devices for implementing the second input circuit 40 being the same as the devices for implementing the first input circuit 30 may mean that the devices for performing the same or corresponding function in the two input circuits are the same.

In the present disclosure, the devices being the “same” may mean that said devices are two separate devices, but at least those of their parameters that are related to generate the excitation signal above, will be used in generating the excitation signal or will affect the generated excitation signal are the same. Alternatively, all parameters of said devices may be the same. It should be noted that the parameters being the same herein doesn't mean that they are absolutely the same in the mathematical sense, but there may be deviations caused by the production process or the manufacturing process, etc. For example, in one embodiment, the parameter being the same may mean that their nominal parameters are the same.

In the embodiments of the present disclosure, the first input circuit 30 and the second input circuit 40 may be any suitable circuit that can be used to generate the excitation signal.

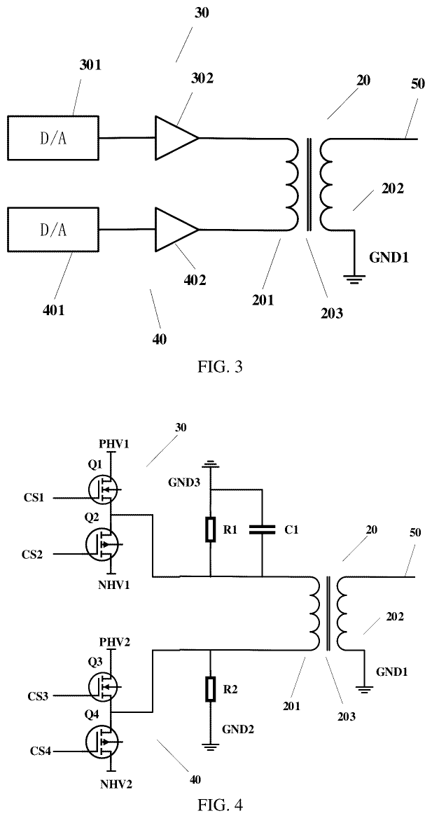

For example, as shown in FIG. 3, in one embodiment, the first input circuit 30 may include a first analog-to-digital converter 301 and a first amplifier 302. The first analog-to-digital converter 301 may receive a transmitting driving signal through its input end and convert the transmitting driving signal into a digital driving signal. The transmitting driving signal may come from a processor or controller of an ultrasound imaging system (e.g., the processor shown in FIG. 1 or another processor or controller not shown in FIG. 1). The transmitting driving signal may be generated by the processor and/or the controller according to current imaging mode and/or imaging parameters of the ultrasound imaging. The input end of the first amplifier 302 is connected to the output end of the first analog-to-digital converter 301, and the output end of the first amplifier 302 is connected to the first end of the primary winding 201 of the transformer 20. The first amplifier 302 can amplify the digital driving signal output by the first analog-to-digital converter 301, and input the amplified digital driving signal to the first end of the primary winding 201.

The second analog-to-digital converter 401 may receive a transmitting driving signal through its input end and convert the transmitting driving signal into a digital driving signal. Similarly, the transmitting driving signal may come from a processor or controller of an ultrasound imaging system (e.g., the processor shown in FIG. 1 or another processor or controller not shown in FIG. 1). The transmitting driving signal may be generated by the processor and/or controller according to current imaging mode and/or imaging parameters of the ultrasound imaging. The input end of the second amplifier 402 is connected to the output end of the second analog-to-digital converter 401, and the output end of the second amplifier 402 is connected to the second end of the primary winding 201 of the transformer 20. The second amplifier 402 can amplify the digital driving signal output by the second analog-to-digital converter 401, and input the amplified digital driving signal to the second end of the primary winding 201.

In this embodiment, the first input circuit 30 and the second input circuit 40 include the analog-to-digital converter that can convert the transmitting driving signals into digital signals, and the digital signals are amplified and inputted into the primary winding of the input transformer, thereby generating the excitation signal that excites the transducers of the ultrasonic probe. In this way, by controlling the analog-to-digital converter, the waveform of the generated excitation signal can be adjusted, so as to obtain a variety of excitation signals with desired waveform. In this art, it may be referred to as “arbitrary waveform transmitting”, and the first input circuit or the second input circuit of this type can be referred to as an arbitrary waveform transmitting circuit.

In one embodiment, the second analog-to-digital converter 401 in the embodiments above may be the same device as the first analog-to-digital converter 301 and the second amplifier 402 may be the same device as the first amplifier 302, which can further improve the symmetry between the generated first excitation signal E1 and the second excitation signal E2 , as mentioned above.

In some embodiments of the present disclosure, a method for generating an excitation signal with the ultrasonic transmitting circuit of the embodiment above is correspondingly provided. The method may include:
controlling the first input circuit 30 to input the first input signal S1 at the first end of the primary winding 201 at a first time, and controlling the second input circuit 40 to input the second input signal S2 at the second end of the primary winding 201 at the first time, such that the transformer 20 outputs the first excitation signal E1 at the first end of the secondary winding 202 according to the first input signal S1 inputted at the first end of the primary winding 201 and the second input signal S2 inputted at the second end of the primary winding 201; and
controlling the first input circuit 30 to input the third input signal S3 at the first end of the primary winding 201 at a second time, and controlling the second input circuit 40 to input the fourth input signal S4 at the second end of the primary winding 201 at the second time, such that the transformer 20 outputs the second excitation signal E2 at the first end of the secondary winding 202 according to the third input signal S3 inputted at the first end of the primary winding 201 and the fourth input signal S4 inputted at the second end of the primary winding 201; where, the first input signal S1 is the same as the fourth input signal S4 , and the second input signal S2 is the same as the third input signal S3 .

In this way, the first excitation signal E1 and the second excitation signal E2 with higher symmetry can be outputted to the transducers of the ultrasonic probe at the first end of the secondary winding 202 of the transformer 20, thereby achieving the symmetrical transmitting with better symmetry.

In other embodiments of the present disclosure, the first input circuit 30 and the second input circuit 40 may include various switches.

For example, as shown in FIG. 4, in one embodiment, the first input circuit 30 may include a first switch Q1 and a second switch Q2.

The first switch Q1 may include a first end, a second end and a control end. The control end may receive the first control signal CS1. The first control signal CS1 controls the first end and second end of the first switch Q1 to be connected or disconnected.

The second switch Q2 may include a first end, a second end and a control end. The control end may receive the second control signal CS2. The second control signal CS1 controls the first end and second end of the second switch Q2 to be connected or disconnected.

The first end of the first switch Q1 is connected to the first high voltage end PHV1 that may receive the first high voltage signal. The second end of the first switch Q1 is connected to the first end of the second switch Q2 and connected to the first end of the primary winding 201 of the transformer 20. The second end of the second switch Q2 is connected to the first low voltage end NHV1 that may receive the first low voltage signal.

It should be noted that the “high voltage” or “low voltage” herein merely indicate the relative relationship of the voltages, but not a specific voltage range. For example, when referring to a “high voltage end”, it merely means that the voltage at this end is higher than the voltage at the “low voltage end”; and, when referring to a “low voltage end”, it merely means that the voltage at this end is lower than the voltage at the “high voltage end”. However, neither of them restricts the voltage at this place to be within a certain “high voltage” or “low voltage” voltage range. Similarly, when referring to a “high voltage signal”, it merely means that the voltage of said voltage signal is higher than the voltage of the “low voltage signal”; and, when referring to a “low voltage signal”, it merely means that the voltage of said voltage signal is lower than the voltage of the “high voltage signal”, and so on. Therefore, the high voltage and low voltage herein may be both positive and negative. Alternatively, the high voltage may be positive and the low voltage may be negative. In the present disclosure, it is not limited, as long as they make the corresponding circuit work normally.

The second input circuit 40 may include a third switch Q3 and a fourth switch Q4.

The third switch Q3 is the same device as the first switch Q1, and includes a first end, a second end and a control end that receives a third control signal CS3. The third control signal CS3 controls the first end and the second end of the third switch Q3 to be connected or disconnected.

The fourth switch Q4 is the same device as the second switch Q2, and includes a first end, a second end and a control end that receives a fourth control signal CS4. The fourth control signal CS4 controls the first end and the second end of the fourth switch Q4 to be connected or disconnected.

The first end of the third switch Q3 is connected to the second high voltage end PHV2. The second high voltage end PHV2 may receive the second high voltage signal that is the same as the first high voltage signal. The second end of the third switch Q3 is connected to the first end of the fourth switch Q4 and connected to the second end of the primary winding 201 of the transformer 20. The second end of the fourth switch Q4 is connected to the second low voltage end NHV2. The second low voltage end NHV2 may receive the second low voltage signal that is the same as the first low voltage signal. Herein, the voltage signal being the “same” may mean that at least the parameters of the voltage signals that are related to generating the excitation signal, will be used in generating the excitation signal or will affect the generated excitation signal are the same. Alternatively, it may also be possible that all parameters of the voltage signals are the same. For example, they may have the same amplitude, phase, frequency, and/or other performance parameters. It should be noted that the same parameters herein are not absolutely the same in the mathematical sense, but there may be deviations caused by devices, circuits, etc. For example, in one embodiment, the same parameter may mean that their nominal parameters are the same.

The first end of the secondary winding of the transformer 20 is connected to the output end 50 of the ultrasonic transmitting circuit. Herein, the first end of the secondary winding being “connected” to the output end 50 of the ultrasonic transmitting circuit may include the first end of the secondary winding being connected to a separate output end 50 through a wire or the first end of the secondary winding itself being the output end 50 of the ultrasonic transmitting circuit. Herein, they are collectively referred to as “connection”.

When the transmitting circuit is working, the first switch Q1 may be controlled to turn on by the first control signal CS1, the second switch Q2 may be controlled to turn off by the second control signal CS2, the third switch Q3 may be controlled to turn off by the third control signal CS3, and the fourth switch Q4 may be controlled to turn on by the fourth control signal CS4. In this way, the passage from the first high voltage end PHV1 to the second low voltage end NHV2 through the first switch Q1, the primary winding 201 of the transformer 20 and the fourth switch Q2 is turned on, such that the first high voltage signal and the second low voltage signal are respectively applied at two ends of the primary winding of the transformer 20, thereby outputting the first excitation signal E1 at the first end of the secondary winding of the transformer.

Thereafter, the first switch Q1 may be controlled to turn off by the first control signal CS1, the second switch Q2 may be controlled to turn on by the second control signal CS2, the third switch Q3 may be controlled to turn on by the third control signal CS3, and the fourth switch Q4 may be controlled to turn off by the fourth control signal CS4. In this way, the passage from the first low voltage end NHV1 to the second high voltage end PHV2 through the second switch Q2, the primary winding 201 of the transformer 20 and the third switch Q3 is turned on, such that the first low voltage signal and the second high voltage signal are respectively applied at tow ends of the primary winding of the transformer 20, thereby outputting the second excitation signal E2 the first end of the secondary winding of the transformer.

In some embodiments of the present disclosure, a method for generating an excitation signal using the ultrasonic transmitting circuit of the embodiments using the switches is provided. The method may include:
controlling the first switch Q1 to turn on, the second switch Q2 to turn off, the third switch Q3 to turn off and the fourth switch Q4 to turn on, such that the passage from the first high voltage end PHV1 to the second low voltage end NHV2 through the first switch Q1, the primary winding 201 of the transformer 20 and the fourth switch Q4 is turned on, thereby outputting the first excitation signal E1 at the first end of the secondary winding 202 of the transformer 20; and
controlling the first switch Q1 to turn off, the second switch Q2 to turn on, the third switch Q3 to turn on and the fourth switch Q4 to turn off, such that the passage from the first low voltage end NHV1 to the second high voltage end PHV2 through the second switch Q2, the primary winding 201 of the transformer 20 and the third switch Q3 is turned on, thereby outputting the second excitation signal E2 at the first end of the secondary winding 202 of the transformer 20.

In the embodiments using the switches above, the first excitation signal is generated with the first switch and the fourth switch, and the second excitation signal is generated with the second switch and the third switch. The first switch and the third switch are the same device, and the second switch and the fourth switch are the same device. That is, the first excitation signal is generated with the two switches, and its performance is affected by the overall performance of the two switches; and the second excitation signal is generated by the same two switches, and its performance is also affected by the overall performance of the same two switches. However, as mentioned above, the existing transmitting circuit usually generate the first excitation signal with one switch (such as the first switch) and generate the second excitation signal with another switch (such as the second switch). In practice, the consistency between the overall performance of the first and fourth switches and the overall performance of the second and third switches is better than that of the first switch and the second switch. Therefore, the first excitation signal and the second excitation signal generated by the circuits and methods of the embodiments of the present disclosure have better symmetry than the excitation signals generated by the existing transmitting circuit.

For example, the first excitation signal is generated by the first high voltage signal and the second low voltage signal via the first switch Q1 and the fourth switch Q4, and the second excitation signal is generated by the first low voltage signal and the second high voltage signal via the second switch Q2 and the third switch Q3. The first switch Q1 and the third switch Q3 are the same device, the second switch Q2 and the fourth switch Q4 are the same device, the second high voltage signal is the same as the first high voltage signal, and the second low voltage signal is the same as the first low voltage signal. That is, the first excitation signal is generated by the high voltage and low voltage signals via Q1+Q4, and its performance is affected by the overall performance of Q1+Q4; the second excitation signal is generated by the same high voltage and low voltage signals via Q2+Q3, and its performance is also affected by the overall performance of Q2+Q3. As mentioned above, the existing transmitting circuit usually generates the first excitation signal with one switch (such as the first switch Q1) and generates the second excitation signal with another switch (such as the second switch Q2). In practice, the consistency between the overall performance of Q1+Q4 and the overall performance of Q2+Q3 is better than the consistency of the performance between the switch Q1 and the switch Q2. Therefore, the first excitation signal and the second excitation signal generated by the circuit or method of this embodiment have better symmetry than the excitation signals generated by the existing transmitting circuit.

For example, in an embodiment where the first and third switches are NMOS transistors and the second and fourth switches are PMOS transistors, or the first and third switches are PMOS transistors and the second and fourth switches are NMOS transistors (see details below), the first excitation signal is generated via a PMOS transistor and an NMOS transistor, and the second excitation signal is generated via a PMOS transistor and an NMOS transistor. Using the existing transmitting circuit, the first excitation signal is generated via a PMOS transistor, and the second excitation signal is generated via an NMOS transistor. Due to the fact that in the current semiconductor process, the PMOS transistor and the NMOS transistor themselves are devices of the two processes, and their performance cannot be completely consistent, the symmetry of the first and second excitation signals generated by the existing transmitting circuit will be poor. However, in the embodiment of the present disclosure, the consistency between the overall performance of one PMOS transistor and one NMOS transistor and the overall performance of another PMOS transistor and another NMOS transistor is better. Therefore, the generated first and second excitation signals have better symmetry.

In the embodiments above, the ultrasonic transmitting circuit may further include a first resistor R1. One end of the first resistor R1 is connected to the first end of the primary winding 201, and the other end is grounded. In the embodiments above, the ultrasonic transmitting circuit may further include a second resistor R2. One end of the second resistor R2 is connected to the second end of the primary winding 201, and the other end is grounded. With the first resistor R1 and/or the second resistor R2, the inductive resonance Q value of the ultrasonic transmitting circuit can be reduced.

In the embodiments above, the ultrasonic transmitting circuit may further include a first capacitor C1. One end of the first capacitor C1 is connected to the first end of the primary winding 201, and the other end is grounded. With the first capacitor C1, asymmetric compensation for the distributed capacitance of the transformer 20 can be realized.

In the embodiments above, the first control signal CS1, the second control signal CS2, the third control signal CS3 and the fourth control signal CS4 may be independently controlled. Alternatively, a part or all of them may be connected together according to the actual situation and be controlled together.

In the embodiments above, the first high voltage signal and the second high voltage signal may be separate or connected together. The first low voltage signal and the second low voltage signal may be separate or connected together.

In the embodiments of the present disclosure, the first, the second, the third and the fourth switches may be any suitable switch devices, such as MOS transistors or triode transistors, or the like.

In one embodiment, as shown in FIG. 4, the first switch Q1 may be an NMOS transistor. The first end of the first switch Q1 is the drain of the NMOS transistor, the second end of the first switch Q1 is the source of the NMOS transistor, and the control end of the first switch Q1 is the gate of the NMOS transistor. The second switch Q2 may be a PMOS transistor. The first end of the second switch Q2 is the drain of the PMOS transistor, the second end of the second switch Q2 is the source of the PMOS transistor, and the control end of the second switch Q2 is the gate of the PMOS transistor. The third switch Q3 may be an NMOS transistor. The first end of the third switch Q3 is the drain of the NMOS transistor, the second end of the third switch Q3 is the source of the NMOS transistor, and the control end of the third switch Q3 is the gate of the NMOS transistor. The fourth switch Q4 may be a PMOS transistor. The first end of the fourth switch Q4 is the drain of the PMOS transistor, the second end of the fourth switch Q4 is the source of the PMOS transistor, and the control end of the fourth switch Q4 is the gate of the PMOS transistor.

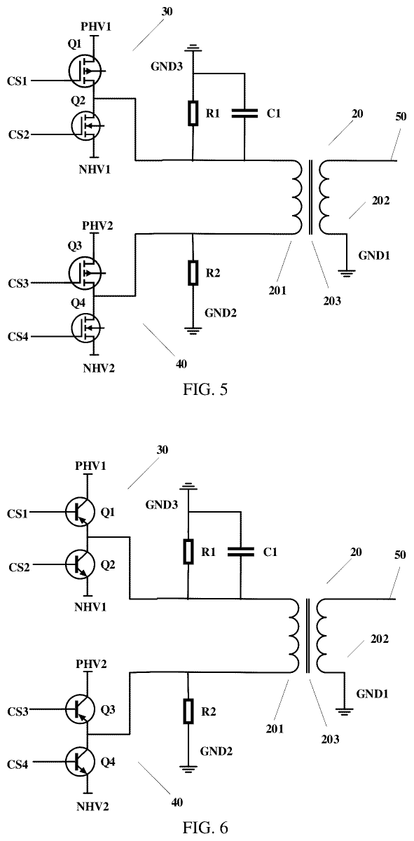

In one embodiment, as shown in FIG. 5, the first switch Q1 may be a PMOS transistor. The first end of the first switch Q1 is the drain of the PMOS transistor, the second end of the first switch Q1 is the source of the PMOS transistor, and the control end of the first switch Q1 is the gate of the PMOS transistor. The second switch Q2 may be an NMOS transistor. The first end of the second switch Q2 is the drain of the NMOS transistor, the second end of the second switch Q2 is the source of the NMOS transistor, and the control end of the second switch Q2 is the gate of the NMOS transistor. The third switch Q3 may be a PMOS transistor. The first end of the third switch Q3 is the drain of the PMOS transistor, the second end of the third switch Q3 is the source of the PMOS transistor, and the control end of the third switch Q3 is the gate of the PMOS transistor. The fourth switch Q4 may be an NMOS transistor. The first end of the fourth switch Q4 is the drain of the NMOS transistor, the second end of the fourth switch Q4 is the source of the NMOS transistor, and the control end of the fourth switch Q4 is the gate of the NMOS transistor.

In the embodiment where the first and third switches are NMOS transistor and the second and fourth switches are PMOS transistor, or the first and third switches are PMOS transistor and the second and fourth switches are NMOS transistor, the first excitation signal is generated via one PMOS transistor and one NMOS transistor, and the second excitation signal is generated also via one PMOS transistor and one NMOS transistor. However, with the existing transmitting circuit, the first excitation signal is generated via the PMOS transistor, and the second excitation signal is generated via the NMOS transistor. Since in the current semiconductor process, the PMOS transistor and the NMOS transistor are actually devices of the two processes and their performance cannot be completely consistent, a difference exists between the parameters of the PMOS transistor and the NMOS transistor. Therefore, the first and second excitation signals generated by them respectively will have poor symmetry. However, in the embodiment of the present disclosure, there is better consistency between the overall performance of one PMOS transistor and one NMOS transistor and the overall performance of the other PMOS transistor and the other NMOS transistor. Therefore, the first and second excitation signals generated thereby have better symmetry.

In one embodiment, as shown in FIG. 6, the first switch Q1 may be a PNP triode transistor. The first end of the first switch Q1 is the collector of the PNP triode transistor, the second end of the first switch Q1 is the emitter of the PNP triode transistor, and the control end of the first switch Q1 is the base of the PNP triode transistor. The second switch Q2 may be an NPN triode transistor. The first end of the second switch Q2 is the collector of the NPN triode transistor, the second end of the second switch Q2 is the emitter of the NPN triode transistor, and the control end of the second switch Q2 is the base of the NPN triode transistor. The third switch Q3 may be a PNP triode transistor. The first end of the third switch Q3 is the collector of the PNP triode transistor, the second end of the third switch Q3 is the emitter of the PNP triode transistor, and the control end of the third switch Q3 is the base of the PNP triode transistor. The fourth switch Q4 may be an NPN triode transistor. The first end of the fourth switch Q4 is the collector of the NPN triode transistor, the second end of the fourth switch Q4 is the emitter of the NPN triode transistor, and the control end of the fourth switch Q4 is the base of the NPN triode transistor.

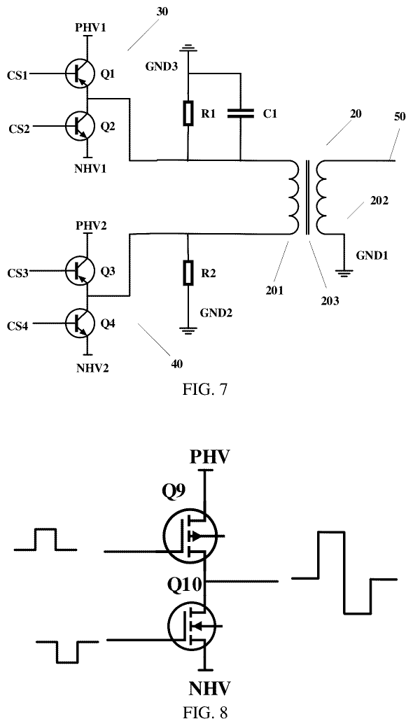

In one embodiment, as shown in FIG. 7, the first switch Q1 may be an NPN triode transistor. The first end of the first switch Q1 is the collector of the NPN triode transistor, the second end of the first switch Q1 is the emitter of the NPN triode transistor, and the control end of the first switch Q1 is the base of the NPN triode transistor. The second switch Q2 may be a PNP triode transistor. The first end of the second switch Q2 is the collector of the PNP triode transistor, the second end of the second switch Q2 is the emitter of the PNP triode transistor, and the control end of the second switch Q2 is the base of the PNP triode transistor. The third switch Q3 may be an NPN triode transistor. The first end of the third switch Q3 is the collector of the NPN triode transistor, the second end of the third switch Q3 is the emitter of the NPN triode transistor, and the control end of the third switch Q3 is the base of the NPN triode transistor. The fourth switch Q4 may be a PNP triode transistor. The first end of the fourth switch Q4 is the collector of the PNP triode transistor, the second end of the fourth switch Q4 is the emitter of the PNP triode transistor, and the control end of the fourth switch Q4 is the base of the PNP triode transistor.

The ultrasonic transmitting circuit in the embodiments above may be implemented by separate devices and electronic circuits, or may be implemented by an integrated circuit.

The present disclosure has been described above through specific embodiments. However, the present disclosure is not limited to these specific embodiments. Those skilled in the art should understand that various modifications, equivalent substitutions or changes, etc. may also be made to the present disclosure, and should be within the protection scope of the present disclosure as long as they do not deviate from the spirit of the present disclosure. In addition, the “one embodiment” described in different places may refer to different embodiments. Of course, all or part of them may be combined in one embodiment.

What is claimed is:

1. An ultrasonic imaging apparatus, comprising:
an ultrasonic probe comprising a transducer;
a transformer comprising at least a primary winding and a secondary winding, wherein the primary winding comprises a first end and a second end, the secondary winding comprises a first end, and the first end of the secondary winding is connected to the transducer of the ultrasonic probe;
a first input circuit connected to the first end of the primary winding; and
a second input circuit connected to the second end of the primary winding;
wherein, the transformer outputs an excitation signal at the first end of the secondary winding according to a signal inputted by the first input circuit at the first end of the primary winding and a signal inputted by the second input circuit at the second end of the primary winding, and the excitation signal excites the transducer connected to the first end of the secondary winding to transmit ultrasonic waves to a target object.

2. The ultrasonic imaging apparatus of claim 1, wherein:
the first input circuit inputs a first input signal at the first end of the primary winding at a first time, the second input circuit inputs a second input signal at the second end of the primary winding at the first time, and the transformer outputs a first excitation signal at the first end of the secondary winding according to the first input signal inputted at the first end of the primary winding and the second input signal inputted at the second end of the primary winding; and
the first input circuit inputs a third input signal at the first end of the primary winding at a second time, the second input circuit inputs a fourth input signal at the second end of the primary winding at the second time, and the transformer outputs a second excitation signal at the first end of the secondary winding according to the third input signal inputted at the first end of the primary winding and the fourth input signal inputted at the second end of the primary winding;
wherein, the first input signal is the same as the fourth input signal, and the second input signal is the same as the third input signal.

3. The ultrasonic imaging apparatus of claim 2, wherein the first input signal is different from the second input signal.

4. The ultrasonic imaging apparatus of claim 1, wherein devices for implementing the second input circuit are the same as devices for implementing the first input circuit.

5. The ultrasonic imaging apparatus of claim 1, wherein the first input circuit comprises:
a first analog-to-digital converter that receives a transmitting driving signal through an input end thereof and converts the transmitting driving signal into a digital driving signal; and
a first amplifier, wherein an input end of the first amplifier is connected to an output end of the first analog-to-digital converter, an output end of the first amplifier is connected to the first end of the primary winding, and the first amplifier amplifies the digital driving signal outputted by the first analog-to-digital converter and inputs the amplified digital driving signal to the first end of the primary winding.

6. The ultrasonic imaging apparatus of claim 3, wherein the second input circuit comprises:
a second analog-to-digital converter that receives a transmitting driving signal through an input end thereof and converts the transmitting driving signal into a digital driving signal; and
a second amplifier, wherein an input end of the second amplifier is connected to an output end of the second analog-to-digital converter, an output end of the second amplifier is connected to the second end of the primary winding, and the second amplifier amplifies the digital driving signal outputted by the second analog-to-digital converter and inputs the amplified digital driving signal to the second end of the primary winding.

7. The ultrasonic imaging apparatus of claim 6, wherein, the second analog-to-digital converter is the same device as a first analog-to-digital converter, and the second amplifier is the same device as a first amplifier.

8. The ultrasonic imaging apparatus of claim 1, wherein the first input circuit comprises:
a first switch comprising a first end, a second end and a control end, wherein the first end of the first switch is connected to a first high voltage end that receives a first high voltage signal, the second end of the first switch is connected to the first end of a second switch and is connected to the first end of the primary winding of the transformer, and the control end of the first switch receives a first control signal; and
the second switch comprising a first end, a second end and a control end, wherein the second end of the second switch is connected to a first low voltage end that receives a first low voltage signal, and the control end of the second switch receives a second control signal.

9. The ultrasonic imaging apparatus of claim 8, wherein the second input circuit comprises:
a third switch comprising a first end, a second end and a control end, wherein the first end of the third switch is connected to a second high voltage end that receives a second high voltage signal, the second high voltage signal is the same as the first high voltage signal, the second end of the third switch is connected to the first end of a fourth switch and is connected to the second end of the primary winding of the transformer, and the control end of the third switch receives a third control signal; and
the fourth switch comprising a first end, a second end and a control end, wherein the second end of the fourth switch is connected to a second low voltage end that receives a second low voltage signal, the second low voltage signal is the same as the first low voltage signal, and the control end of the fourth switch receives a fourth control signal.

10. The ultrasonic imaging apparatus of claim 9, wherein the third switch is the same device as the first switch, and the fourth switch is the same device as the second switch.

11. The ultrasonic imaging apparatus of claim 8, wherein:
the first switch is an NMOS transistor, the first end of the first switch is a drain of the NMOS transistor, the second end of the first switch is a source of the NMOS transistor, and the control end of the first switch is a gate of the NMOS transistor; and
the second switch is a PMOS transistor, the first end of the second switch is a drain of the PMOS transistor, the second end of the second switch is a source of the PMOS transistor, and the control end of the second switch is a gate of the PMOS transistor.

12. The ultrasonic imaging apparatus of claim 9, wherein:
the third switch is an NMOS transistor, the first end of the third switch is a drain of the NMOS transistor, the second end of the third switch is a source of the NMOS transistor, and the control end of the third switch is a gate of the NMOS transistor; and
the fourth switch is a PMOS transistor, the first end of the fourth switch is a drain of the PMOS transistor, the second end of the fourth switch is a source of the PMOS transistor, and the control end of the fourth switch is a gate of the PMOS transistor.

13. The ultrasonic imaging apparatus of claim 8, wherein:
the first switch is a PMOS transistor, the first end of the first switch is a drain of the PMOS transistor, the second end of the first switch is a source of the PMOS transistor, and the control end of the first switch is a gate of the PMOS transistor; and
the second switch is an NMOS transistor, the first end of the second switch is a drain of the NMOS transistor, the second end of the second switch is a source of the NMOS transistor, and the control end of the second switch is a gate of the NMOS transistor.

14. The ultrasonic imaging apparatus of claim 9, wherein:
the third switch is a PMOS transistor, the first end of the third switch is a drain of the PMOS transistor, the second end of the third switch is a source of the PMOS transistor, and the control end of the third switch is a gate of the PMOS transistor; and
the fourth switch is an NMOS transistor, the first end of the fourth switch is a drain of the NMOS transistor, the second end of the fourth switch is a source of the NMOS transistor, and the control end of the fourth switch is a gate of the NMOS transistor.

15. The ultrasonic imaging apparatus of claim 8, wherein:
the first switch is a PNP triode transistor, the first end of the first switch is a collector of the PNP triode transistor, the second end of the first switch is an emitter of the PNP triode transistor, and the control end of the first switch is a base of the PNP triode transistor; and
the second switch is an NPN triode transistor, the first end of the second switch is a collector of the NPN triode transistor, the second end of the second switch is an emitter of the NPN triode transistor, and the control end of the second switch is a base of the NPN triode transistor.

16. The ultrasonic imaging apparatus of claim 9, wherein:
the third switch is a PNP triode transistor, the first end of the third switch is a collector of the PNP triode transistor, the second end of the third switch is an emitter of the PNP triode transistor, and the control end of the third switch is a base of the PNP triode transistor; and
the fourth switch is an NPN triode transistor, the first end of the fourth switch is a collector of the NPN triode transistor, the second end of the fourth switch is an emitter of the NPN triode transistor, and the control end of the fourth switch is a base of the NPN triode transistor.

17. The ultrasonic imaging apparatus of claim 8, wherein:
the first switch is an NPN triode transistor, the first end of the first switch is a collector of the NPN triode transistor, the second end of the first switch is an emitter of the NPN triode transistor, and the control end of the first switch is a base of the NPN triode transistor; and
the second switch is a PNP triode transistor, the first end of the second switch is a collector of the PNP triode transistor, the second end of the second switch is an emitter of the PNP triode transistor, and the control end of the second switch is a base of the PNP triode transistor.

18. The ultrasonic imaging apparatus of claim 9, wherein:
the third switch is an NPN triode transistor, the first end of the third switch is a collector of the NPN triode transistor, the second end of the third switch is an emitter of the NPN triode transistor, and the control end of the third switch is a base of the NPN triode transistor; and
the fourth switch is a PNP triode transistor, the first end of the fourth switch is a collector of the PNP triode transistor, the second end of the fourth switch is an emitter of the PNP triode transistor, and the control end of the fourth switch is a base of the PNP triode transistor.

19. An ultrasonic transmitting circuit comprising:
a transformer comprising at least a primary winding and a secondary winding, wherein, the primary winding comprises a first end and a second end, the secondary winding comprises a first end, and the first end of the secondary winding is connected to a transducer of an ultrasonic probe;
a first input circuit that is connected to the first end of the primary winding; and
a second input circuit that is connected to the second end of the primary winding;
wherein, the transformer outputs an excitation signal at the first end of the secondary winding according to a signal inputted by the first input circuit at the first end of the primary winding and a signal inputted by the second input circuit at the second end of the primary winding, wherein the excitation signal excites the transducer connected to the first end of the secondary winding to transmit ultrasonic waves to a target object.

20. A method for generating an excitation signal with an ultrasonic transmitting circuit, wherein:
the ultrasonic transmitting circuit comprises:
a transformer comprising at least a primary winding and a secondary winding, wherein, the primary winding comprises a first end and a second end, the secondary winding comprises a first end, and the first end of the secondary winding is connected to a transducer of an ultrasonic probe;
a first input circuit connected to the first end of the primary winding; and
a second input circuit connected to the second end of the primary winding;
wherein, the transformer outputs an excitation signal at the first end of the secondary winding according to a signal inputted by the first input circuit at the first end of the primary winding and a signal inputted by the second input circuit at the second end of the primary winding;
the method comprises:
controlling the first input circuit to input a first input signal at the first end of the primary winding at a first time, and controlling the second input circuit to input a second input signal at the second end of the primary winding at the first time, such that the transformer outputs a first excitation signal at the first end of the secondary winding according to the first input signal inputted at the first end of the primary winding and the second input signal inputted at the second end of the primary winding; and
controlling the first input circuit to input a third input signal at the first end of the primary winding at a second time, and controlling the second input circuit to input a fourth input signal at the second end of the primary winding at the second time, such that the transformer outputs a second excitation signal at the first end of the secondary winding according to the third input signal inputted at the first end of the primary winding and the fourth input signal inputted at the second end of the primary winding;
wherein, the first input signal is the same as the fourth input signal, and the second input signal is the same as the third input signal.
